(19) United States
(12) Patent Application Publication
(10) Pub. No.: US 2009/0095200 A1
(43) Pub. Date: Apr. 16, 2009

(54) Modified colored pigments

(76) Inventors: James A. Belmont, Acton, MA (US); Elizabeth G. Burns, Windham, NH (US)

Correspondence Address: Michelle B. Lando, Esq.;CABOT CORPORATION, Law Department, 157 Concord Road, Billerica, MA, 01821, US

(21) Appl. No.: 11/973,687

(22) Filed: Oct. 10, 2007

(51) Int. Cl. C09D 11/10; C07C 57/145; C07C 57/15; C07D 307/33; C08F 12/08; C08F 16/00; C08F 20/00; C08F 26/02

(52) U.S. Cl. 106/31.85; 106/31.6; 525/451; 526/301; 526/317.1; 526/332; 526/346; 526/72; 528/322; 528/363; 528/367; 528/422; 549/477; 562/595; 562/598

(57) ABSTRACT

The present invention relates to a modified colored pigment comprising a colored pigment having an attached organic group. The organic group is attached to the colored pigment via at least one carbon atom of a C—C single bond or double bond, wherein the C—C single bond or double bond is not a component of an aromatic system. The organic group further comprises at least one activating group on at least one carbon atom of the C—C single bond or double bond. A method of preparing a modified colored pigment is also disclosed, as are aqueous and non-aqueous dispersions, e.g., including ink jet inks, comprising the modified colored pigments.

### BACKGROUND OF THE INVENTION

1. Field of the Invention

The present invention relates to modified colored pigments as well as to dispersions and inkjet ink compositions comprising these modified colored pigments.

2. Description of the Related Art

Modified colored pigment products are well known in various applications. For example, the attachment of various organic groups to colored pigments is known to improve the dispersibility of the pigment in a liquid vehicle (e.g., an aqueous medium). Such improved dispersibility advantageously promotes uniform distribution of the colored pigment throughout the composition of interest.

U.S. Pat. Nos. 5,837,045 and 5,922,118 to Johnson et al. disclose modified colored pigments having at least one attached organic group wherein the organic group comprises (i) at least one aromatic group or a C₁-C₁₂ alkyl group and (ii) at least one ionic or ionizable group. The Johnson patents further disclose aqueous and non-aqueous dispersions containing the modified colored pigment suitable for use in a variety of applications (e.g., including inks, toners, paints, adhesives, coatings, and plastics). The Johnson patents also disclose methods for preparing the above described modified colored pigments.

U.S. Pat. No. 6,660,075 discloses carbon blacks having attached organic groups, wherein the organic groups are connected to the carbon black by one or two carbon atoms of a C—C single bond or double bond that is not a component of an aromatic system. Further disclosure is made of using the blacks in fillers, reinforcing fillers, UV stabilizers, conductance blacks, or pigments.

While modified colored pigments may be utilized in various applications, such as in ink jet inks, there remains a need for modified pigments having further improved performance properties, thereby providing advantageous alternatives to previous modified pigments.

### SUMMARY OF THE INVENTION

The present invention relates to a modified colored pigment having at least one attached organic group. The organic group is attached to the colored pigment via at least one carbon atom of a C—C single bond or double bond that is not a component of an aromatic system. The organic group further comprises at least one activating group on at least one carbon atom of the C—C single bond or double bond. The colored pigment can be any type of organic pigment and does not include carbon black pigments. Preferred pigments include phthalocyanine blues and quinacridones.

The invention further relates to aqueous and non-aqueous dispersions comprising a liquid vehicle and the modified colored pigment described above. Such dispersions may be utilized in substantially any suitable composition, for example, including plastics, rubbers, papers, textiles, coatings, paints, toners, adhesives, latexes, and inks.

The foregoing has outlined rather broadly the features of the present invention in order that the detailed description that follows may be better understood. Additional features and advantages of the invention will be described hereinafter which form the subject of the claims of the invention. It will be understood that both the foregoing general description and the following detailed description are exemplary and explanatory only and are intended to provide further explanation of the present invention, as claimed.

### DETAILED DESCRIPTION OF THE INVENTION

The present invention relates to a modified colored pigment having at least one attached organic group. The organic group is attached to the colored pigment via at least one carbon atom of a C—C single bond or double bond, wherein the C—C single bond or double bond is not a component of an aromatic system. The organic group further comprises at least one activating group on at least one carbon atom of the C—C single bond or double bond.

The term colored pigment, as used herein, refers to any organic colored pigment. Colored pigments in accordance with the present invention may include substantially any type of organic pigment, such as organic black pigments and other organic colored pigments including blue, brown, cyan, green, white, violet, magenta, red, orange, or yellow pigments. Colored pigments in accordance with the invention do not include carbon black pigments. Suitable classes of organic black pigments include aniline blacks and perylene blacks. A representative example of aniline black includes Pigment Black 1. Representative examples of perylene blacks include Pigment Black 31 and Pigment Black 32.

Suitable classes of colored pigments also include, for example, anthraquinones, phthalocyanine blues, phthalocyanine greens, diazos, monoazos, pyranthrones, perylenes, heterocyclic yellows, quinacridones, quinolonoquinolones, and (thio)indigoids. Representative examples of phthalocyanine blues include copper phthalocyanine blue and derivatives thereof (e.g., the Pigment Blue 15 series). Representative examples of phthalocyanine greens include polychloro copper phthalocyanine green and derivatives thereof (e.g., Pigment Green 7 and Pigment Green 36). Representative examples of quinacridones include Pigment Orange 48, Pigment Orange 49, Pigment Red 122, Pigment Red 192, Pigment Red 202, Pigment Red 206, Pigment Red 207, Pigment Red 209, Pigment Violet 19 and Pigment Violet 42. Representative examples of anthraquinones include Pigment Red 43, Pigment Red 194 (Perinone Red), Pigment Red 216 (Brominated Pyanthrone Red) and Pigment Red 226 (Pyranthrone Red). Representative examples of perylenes include Pigment Red 123 (Vermillion), Pigment Red 149 (Scarlet), Pigment Red 179 (Maroon), Pigment Red 190 (Red), Pigment Violet 19, Pigment Red 189 (Yellow Shade Red) and Pigment Red 224. Representative examples of thioindigoids include Pigment Red 86, Pigment Red 87, Pigment Red 88, Pigment Red 181, Pigment Red 198, Pigment Violet 36, and Pigment Violet 38. Representative examples of heterocyclic yellows include Pigment Yellow 1, Pigment Yellow 3, Pigment Yellow 12, Pigment Yellow 13, Pigment Yellow 14, Pigment Yellow 17, Pigment Yellow 65, Pigment Yellow 73, Pigment Yellow 74, Pigment Yellow 151, Pigment Yellow 117, Pigment Yellow 128 and Pigment Yellow 138. Such pigments are commercially available in either powder or press cake form from a number of sources including, BASF Corporation, Engelhard Corporation, Sun Chemical Corporation, Clariant, and Dianippon Ink and Chemicals (DIC). Examples of other suitable colored pigments are described in the Colour Index, 3rd edition (The Society of Dyers and Colourists, 1982). Mixtures of different pigments may, of course, be used, either as a blend of separate pigments or a pigment which comprises multiple colorants. Preferred classes of pigments include phthalocyanine blues and quinacridones.

The pigment can have a wide range of BET surface areas, as measured by nitrogen adsorption, depending on the desired properties of the pigment. Preferably, the pigments have a BET surface area between about 10 m²/g and about 1500 m²/g, more preferably between about 20 m²/g and about 600 m²/g and most preferably between about 50 m²/g and about 300 m²/g. If the desired surface area is not readily available for the desired application, it is also well recognized by those skilled in the art that the pigment may be subjected to conventional size reduction or comminution techniques, such as ball or jet milling or sonication, to reduce the pigment to a smaller particle size, if desired. Also, the pigment can have a wide variety of crystallite sizes known in the art. For example, the pigment may have a crystallite size of between about 5 nm to about 100 nm, including about 10 nm to about 80 nm and 15 nm to about 50 nm. In addition, the pigment can also have a wide range of oil absorption values, which is a measure of the structure of the pigment. For example, the pigment may be an organic colored pigment having an oil absorption value (as described in ISO 787 T5) of from about 5 to 150 mL/100 g, including from about 10 to 100 mL/100 g and from about 20 to 80 mL/100 g.

The pigment may also be a pigment that has been oxidized using an oxidizing agent in order to introduce ionic and/or ionizable groups onto the surface. In addition, pigments prepared using other surface modification methods, such as chlorination, sulfonation, and sulfonylation, may also be used.

The organic group is attached to the colored pigment via at least one carbon atom of a C—C single bond or double bond, wherein the C—C single bond or double bond is not a component of an aromatic system. The organic group may be attached to the colored pigment by reacting the pigment with organic compounds having a C—C double or triple bond and at least one activating group on at least one carbon atom of the C—C double or triple bond. The resulting modified pigment comprises at least one activating group on at least one carbon atom of the C—C single bond or double bond. Electron withdrawing activating groups are generally preferred. Suitable activating groups include, for example, acyl, formyl, carboxyl, alkoxycarbonyl, aryloxycarbonyl, acyloxycarbonyl, cyano, carbamoyl, alkylcarbamoyl, arylcarbamoyl, acylcarbamoyl, alkylsulfonyl, arylsulfonyl, alkyloxysulfonyl, aryloxysulfonyl, alkylsulfinyl, arylsulfinyl, phosphonyl, alkoxyphosphonyl, and aryloxyphosphonyl groups. Preferred activating groups include: carboxyl, alkoxycarbonyl, aryloxycarbonyl, acyloxycarbonyl, carbamoyl, alkylcarbamoyl, arylcarbamoyl, and acylcarbamoyl groups.

More preferred activating groups include —CO—R⁵, —CHO, —COOR⁵, —COOH, —CN, —SO₂R⁵, and —CO—X—CO—, wherein X is selected from the group consisting of: O and N—R⁵. R⁵ is selected from the group consisting of: alkyl, Y-functionalized alkyl, polymers, cyclic organic groups, aryl, and Y-functionalized aryl of the form Ar—Yₙ; wherein n is from 1 to 5 and Y is selected from the group consisting of —OH, —OR, —SH, —SR, —SO₃H, —SO₃⁻M⁺, —SO₂R, —B(OH)₂, —O(CH₂—CH₂—O)ₘ—H, —COOH, —COO⁻M⁺, —NH₂, —NR₂, —N((CH₂—CH₂—O)ₙH)₂, —CON((CH₂—CH₂—O)ₙH)₂, trialkoxysilyl, perfluoroalkyl, alkyl, —NH₃⁺Q⁻, —NR₃⁺Q⁻, —SO₂—NR₂, —NO₂, —Cl, —COR, —COOR, —NO₂, —SSR, —SCN, —CN, —PO₃H₂, and —PO₃⁻²M⁺₂; wherein M⁺ is an alkali metal ion or an alkaline earth metal ion; Q⁻ is a halide ion, a nitrate ion, a carboxylate ion (such as an acetate ion), or a sulfate ion; m is from 1 to 50, and R is selected from the group consisting of: H, alkyl, aryl, functionalized alkyl, functionalized aryl, and polymers. Preferred polymers for R and R⁵ include polyamines, polyalkylene oxides, polyalkylene glycols, polyols, poly(meth)acrylates, proteins, polyamino acids, polyureas, polyurethanes, polyureas/urethanes, polyesters, polyethers, polyvinylethers, polyamides, polyimides, polyolefins, polyolefin-acrylates, polystyrenes (including styrene(meth)acrylate copolymers), salts thereof, and derivatives thereof.

Another aspect of the invention is a method of producing a modified colored pigment having at least one attached organic group. The method comprises the step of reacting a colored pigment with an organic compound having a C—C double or triple bond and at least one activating group on at least one carbon atom of the C—C double or triple bond. The reaction product comprises a colored pigment having at least one attached organic group as described above. Substantially any suitable organic compound having a C—C double or triple bond and at least one activating group on at least one carbon atom of the C—C double or triple bond may be utilized. Such organic compounds may be generically referred to as alkyne (triple bond) derivatives or alkene (double bond) derivatives. Alkyne derivatives may be represented by the formula R¹C≡CR² and alkene derivatives may be represented by the formula R¹R³C═CR²R⁴. R¹ may include, for example, —CO—R⁵, —CHO, —COOR⁵, —COOH, —CN, and —SO₂R⁵. R², R³, and R⁴ may include R¹, H, alkyl, or aryl. R⁵ may be any of those described above and may include alkyl, aryl, or functionalized alkyl or aryl such as, ω-carboxyalkyl, carboxyaryl, HO₃S-alkyl-, H₂N-alkyl-, H₂N—SO₂-alkyl-, HO₃S-aryl-, H₂N-aryl-, H₂N—SO₂-aryl-, and similar groups.

Suitable organic compounds may also include maleic anhydride and maleic acid derivatives. Maleic acid derivatives may be represented by the formula C₄H₂O₂X where X may include O and N—R⁵. R⁵ may be as defined above. R⁵ may also include an aliphatic group, a cyclic organic group, or an organic compound with an aliphatic part and a cyclic part. R⁵ may further be substituted or unsubstituted, branched or unbranched, or include an aliphatic group, e.g., groups of alkanes, alkenes, alcohols, ethers, aldehydes, ketones, carboxylic acids, esters, and hydrocarbons. R⁵ may still further include a cyclic compound (e.g., alicyclic hydrocarbons such as cycloalkyls or cycloalkenyls), a heterocyclic compound (e.g., pyrrolidinyl-, pyrrolinyl-, piperidinyl, or morpholinyl), an aryl group (e.g. phenyl, naphthyl, or anthracenyl), or a heteroaryl group (e.g., imidizoyl, pyrazoyl, pyridinyl, thienyl, thiazolyl, furyl, or indolyl).

Preferred organic compounds include maleic anhydride, maleic acid, fumaric acid, acetylene dicarboxylic acid, and derivatives thereof. Preferred derivatives of maleic acid, fumaric acid, and acetylene dicarboxylic acid include carboxylic acids, dicarboxylic acids, esters, amides, and imides. Maleic anhydride is a most preferred organic compound.

The above described organic compounds have been surprisingly found to react with and modify organic colored pigments. As described in more detail below (in the Examples), organic colored pigments may be reacted with a sufficient amount of an organic compound such that the colored pigment is dispersible in an aqueous medium. U.S. Pat. No. 6,660,075 discloses carbon blacks reacted with the above described organic compounds. Organic colored pigments have very different surface reactive sites and surface reactivities and would therefore not be expected to react with the same types of organic compounds as do carbon blacks.

The above described modified colored pigment may be further reacted with a nucleophilic polymer to form a polymer modified pigment. Substantially any suitable nucleophilic polymer may be utilized. Exemplary polymers that comprise or could be modified to comprise a nucleophile include polyamines, polyalkylene oxides, polyalkylene glycols, polyols, poly(meth)acrylates, proteins, polyamino acids, polyureas, polyurethanes, polyureas/urethanes, polyesters, polyethers, polyvinylethers, polyamides, polyimides, polyolefins, polyolefin-acrylates, polystyrenes (such as styrene(meth)acrylate copolymers), salts thereof, and derivatives thereof.

The modified pigments of the present invention may be used in a variety of applications, including, for example, plastic compositions, aqueous or non-aqueous inks, aqueous or non-aqueous coatings, rubber compositions, paper compositions, toners, and textile compositions. In particular, these pigments may be used in aqueous compositions, including, for example, automotive and industrial coatings, paints, adhesives, latexes, and inks. The pigments have been found to be most useful in ink compositions, especially inkjet inks. Thus, the present invention further relates to dispersions and inkjet ink compositions comprising a vehicle and a modified pigment. The vehicle can be either an aqueous or non-aqueous liquid vehicle, but is preferably a vehicle that contains water. Thus, the vehicle is preferably an aqueous vehicle, and the dispersion and inkjet ink compositions are aqueous compositions. More preferably the vehicle contains greater than 50% water and includes, for example, water or mixtures of water with water miscible solvents such as alcohols.

Inkjet ink compositions in accordance with the present invention can be formed with a minimum of additional components (additives and/or cosolvents) and processing steps. However, suitable additives may be incorporated in order to impart a number of desired properties while maintaining the stability of the compositions. For example, surfactants and/or dispersants, humectants, drying accelerators, penetrants, biocides, binders, and pH control agents, as well as other additives known in the art, may be added. The amount of a particular additive will vary depending on a variety of factors but generally ranges between 0% and 40%.

Dispersing agents (surfactants and/or dispersants) may be added to further enhance the colloidal stability of the composition or to change the interaction of the ink with either the printing substrate, such as printing paper, or with the ink printhead. Various anionic, cationic and nonionic dispersing agents can be used in conjunction with the ink composition of the present invention, and these may be in solid form or as a water solution.

Representative examples of anionic dispersants or surfactants include, but are not limited to, higher fatty acid salts, higher alkyldicarboxylates, sulfuric acid ester salts of higher alcohols, higher alkyl-sulfonates, alkylbenzenesulfonates, alkylnaphthalene sulfonates, naphthalene sulfonates (Na, K, Li, Ca, etc.), formalin polycondensates, condensates between higher fatty acids and amino acids, dialkylsulfosuccinic acid ester salts, alkylsulfosuccinates, naphthenates, alkylether carboxylates, acylated peptides, α-olefin sulfonates, N-acrylmethyl taurine, alkylether sulfonates, secondary higher alcohol ethoxysulfates, polyoxyethylene alkylphenylether sulfates, monoglycylsulfates, alkylether phosphates and alkyl phosphates, alkyl phosphonates and bisphosphonates, included hydroxylated or aminated derivatives. For example, polymers and copolymers of styrene sulfonate salts, unsubstituted and substituted naphthalene sulfonate salts (e.g. alkyl or alkoxy substituted naphthalene derivatives), aldehyde derivatives (such as unsubstituted alkyl aldehyde derivatives including formaldehyde, acetaldehyde, propylaldehyde, and the like), maleic acid salts, and mixtures thereof may be used as the anionic dispersing aids. Salts include, for example, Na⁺, Li⁺, K⁺, Cs⁺, Rb⁺, and substituted and unsubstituted ammonium cations. Specific examples include, but are not limited to, commercial products such as Versa® 4, Versa® 7, and Versa® 77 (National Starch and Chemical Co.); Lomar® D (Diamond Shamrock Chemicals Co.); Daxad®19 and Daxad® K (W. R. Grace Co.); and Tamol® SN (Rohm & Haas). Representative examples of cationic surfactants include aliphatic amines, quaternary ammonium salts, sulfonium salts, phosphonium salts and the like.

Representative examples of nonionic dispersants or surfactants that can be used in ink jet inks of the present invention include fluorine derivatives, silicone derivatives, acrylic acid copolymers, polyoxyethylene alkyl ether, polyoxyethylene alkylphenyl ether, polyoxyethylene secondary alcohol ether, polyoxyethylene styrol ether, ethoxylated acetylenic diols (such as Surfynol® 420, Surfynol® 440, and Surfynol® 465, available from Air Products), polyoxyethylene lanolin derivatives, ethylene oxide derivatives of alkylphenol formalin condensates, polyoxyethylene polyoxypropylene block polymers, fatty acid esters of polyoxyethylene polyoxypropylene alkylether polyoxyethylene compounds, ethylene glycol fatty acid esters of polyethylene oxide condensation type, fatty acid monoglycerides, fatty acid esters of polyglycerol, fatty acid esters of propylene glycol, cane sugar fatty acid esters, fatty acid alkanol amides, polyoxyethylene fatty acid amides and polyoxyethylene alkylamine oxides. For example, ethoxylated monoalkyl or dialkyl phenols may be used, such as Igepal® CA and CO series materials (Rhone-Poulenc Co.), Brij® Series materials (ICI Americas, Inc.), and Triton® series materials (Union Carbide Company). These nonionic surfactants or dispersants can be used alone or in combination with the aforementioned anionic and cationic dispersants.

The dispersing agents may also be a natural polymer or a synthetic polymer dispersant. Specific examples of natural polymer dispersants include proteins such as glue, gelatin, casein and albumin; natural rubbers such as gum arabic and tragacanth gum; glucosides such as saponin; alginic acid, and alginic acid derivatives such as propyleneglycol alginate, triethanolamine alginate, and ammonium alginate; and cellulose derivatives such as methyl cellulose, carboxymethyl cellulose, hydroxyethyl cellulose and ethylhydroxy cellulose. Specific examples of polymeric dispersants, including synthetic polymeric dispersants, include polyvinyl alcohols, such as Elvanols from DuPont, Celvoline from Celanese, polyvinylpyrrolidones such as Luvatec from BASF, Kollidon and Plasdone from ISP, and PVP-K, Glide, acrylic or methacrylic resins (often written as “(meth)acrylic”) such as poly(meth)acrylic acid, Ethacryl line from Lyondell, Alcosperse from Alco, acrylic acid-(meth)acrylonitrile copolymers, potassium (meth)acrylate-(meth)acrylonitrile copolymers, vinyl acetate-(meth)acrylate ester copolymers and (meth)acrylic acid-(meth)acrylate ester copolymers; styrene-acrylic or methacrylic resins such as styrene-(meth)acrylic acid copolymers, such as the Joncryl line from BASF, Carbomers from Noveon, styrene-(meth)acrylic acid-(meth)acrylate ester copolymers, such as the Joncryl polymers from BASF, styrene-α-methylstyrene-(meth)acrylic acid copolymers, styrene-α-methylstyrene-(meth)acrylic acid-(meth)acrylate ester copolymers; styrene-maleic acid copolymers; styrene-maleic anhydride copolymers, such as the SMA polymers from Sartomer, vinyl naphthalene-acrylic or methacrylic acid copolymers; vinyl naphthalene-maleic acid copolymers; and vinyl acetate copolymers such as vinyl acetate-ethylene copolymer, vinyl acetate-fatty acid vinyl ethylene copolymers, vinyl acetate-maleate ester copolymers, vinyl acetate-crotonic acid copolymer and vinyl acetate-acrylic acid copolymer; and salts thereof. Polymers, such as those listed above, variations and related materials, that can be used for dispersants and additives in inkjet inks are included in the Tego products from Degussa, the Ethacryl products from Lyondell, the Joncryl polymers from BASF, the EFKA dispersants from Ciba, and the Disperbyk and Byk dispersants from BYK Chemie.

Humectants and water soluble organic compounds may also be added to the inkjet ink composition of the present invention, particularly for the purpose of preventing clogging of the nozzle as well as for providing paper penetration (penetrants), improved drying (drying accelerators), and anti-cockling properties. Specific examples of humectants and other water soluble compounds that may be used include low molecular-weight glycols such as ethylene glycol, diethylene glycol, triethylene glycol, tetraethylene glycol and dipropylene glycol; diols containing from about 2 to about 40 carbon atoms, such as 1,3-pentanediol, 1,4-butanediol, 1,5-pentanediol, 1,4-pentanediol, 1,6-hexanediol, 1,5-hexanediol, 2,6-hexanediol, neopentylglycol (2,2-dimethyl-1,3-propanediol), 1,3-propanediol, 1,4-butanediol, 1,5-pentanediol, 1,6-hexanediol, 1,2,6-hexanetriol, poly(ethylene-co-propylene) glycol, and the like, as well as their reaction products with alkylene oxides, including ethylene oxides, including ethylene oxide and propylene oxide; triol derivatives containing from about 3 to about 40 carbon atoms, including glycerine, trimethylolpropane, 1,3,5-pentanetriol, 1,2,6-hexanetriol, and the like as well as their reaction products with alkylene oxides, including ethylene oxide, propylene oxide, and mixtures thereof; neopentylglycol, (2,2-dimethyl-1,3-propanediol), and the like, as well as their reaction products with alkylene oxides, including ethylene oxide and propylene oxide in any desirable molar ratio to form materials with a wide range of molecular weights; thiodiglycol; pentaerythritol and lower alcohols such as ethanol, propanol, iso-propyl alcohol, n-butyl alcohol, sec-butyl alcohol, and tert-butyl alcohol, 2-propyn-1-ol (propargyl alcohol), 2-buten-1-ol, 3-buten-2-ol, 3-butyn-2-ol, and cyclopropanol; amides such as dimethyl formaldehyde and dimethyl acetamide; ketones or ketoalcohols such as acetone and diacetone alcohol; ethers such as tetrahydrofuran and dioxane; cellosolves such as ethylene glycol monomethyl ether and ethylene glycol monoethyl ether, triethylene glycol monomethyl (or monoethyl)ether; carbitols such as diethylene glycol monomethyl ether, diethylene glycol monoethyl ether, and diethylene glycol monobutyl ether; lactams such as 2-pyrrolidone, N-methyl-2-pyrrolidone and ε-caprolactam; urea and urea derivatives; inner salts such as betaine, and the like; thio (sulfur) derivatives of the aforementioned materials including 1-butanethiol; t-butanethiol 1-methyl-1-propanethiol, 2-methyl-1-propanethiol; 2-methyl-2-propanethiol; thiocyclopropanol, thioethyleneglycol, thiodiethyleneglycol, trithio- or dithio-diethyleneglycol, and the like; hydroxyamide derivatives, including acetylethanolamine, acetylpropanolamine, propylcarboxyethanolamine, propylcarboxy propanolamine, and the like; reaction products of the aforementioned materials with alkylene oxides; and mixtures thereof. Additional examples include saccharides such as maltitol, sorbitol, gluconolactone and maltose; polyhydric alcohols such as trimethylol propane and trimethylol ethane; N-methyl-2-pyrrolidone; 1,3-dimethyl-2-imidazolidinone; sulfoxide derivatives containing from about 2 to about 40 carbon atoms, including dialkylsulfides (symmetric and asymmetric sulfoxides) such as dimethylsulfoxide, methylethylsulfoxide, alkylphenyl sulfoxides, and the like; and sulfone derivatives (symmetric and asymmetric sulfones) containing from about 2 to about 40 carbon atoms, such as dimethylsulfone, methylethylsulfone, sulfolane (tetramethylenesulfone, a cyclic sulfone), dialkyl sulfones, alkyl phenyl sulfones, dimethylsulfone, methylethylsulfone, diethylsulfone, ethylpropylsulfone, methylphenylsulfone, methylsulfolane, dimethylsulfolane, and the like. Such materials may be used alone or in combination.

Biocides and/or fungicides may also be added to the inkjet ink composition of the present invention. Biocides are important in preventing bacterial growth since bacteria are often larger than ink nozzles and can cause clogging as well as other printing problems. Examples of useful biocides include, but are not limited to, benzoate or sorbate salts, and isothiazolinones.

Various polymeric binders can also be used in conjunction with the inkjet ink composition of the present invention to adjust the viscosity of the composition as well as to provide other desirable properties. Suitable polymeric binders include, but are not limited to, water soluble polymers and copolymers such as gum arabic, polyacrylate salts, polymethacrylate salts, polyvinyl alcohols (Elvanols from DuPont, Celvoline from Celanese), hydroxypropylenecellulose, hydroxyethylcellulose, polyvinylpyrrolidinone (such as Luvatec from BASF, Kollidon and Plasdone from ISP, and PVP-K, Glide), polyvinylether, starch, polysaccharides, polyethyleneimines with or without being derivatized with ethylene oxide and propylene oxide including the Discole® series (DKS International); the Jeffamine® series (Huntsman); and the like. Additional examples of water-soluble polymer compounds include various dispersants or surfactants described above, including, for example, styrene-acrylic acid copolymers (such as the Joncryl line from BASF, Carbomers from Noveon), styrene-acrylic acid-alkyl acrylate terpolymers, styrene-methacrylic acid copolymers (such as the Joncryl line from BASF), styrene-maleic acid copolymers (such as the SMA polymers from Sartomer), styrene-maleic acid-alkyl acrylate terpolymers, styrene-methacrylic acid-alkyl acrylate terpolymers, styrene-maleic acid half ester copolymers, vinyl naphthalene-acrylic acid copolymers, alginic acid, polyacrylic acids or their salts and their derivatives. In addition, the binder may be added or present in dispersion or latex form. For example, the polymeric binder may be a latex of acrylate or methacrylate copolymers (such as NeoCryl materials from NSM Neoresins, the AC and AS polymers from Alberdingk-Boley) or may be a water dispersible polyurethane (such as ABU from Alberdingk-Boley) or polyester (such as AQ polymers from Eastman Chemical). Polymers, such as those listed above, variations and related materials, that can be used for binders in inkjet inks are included in the Ethacryl products from Lyondell, the Joncryl polymers from BASF, the NeoCryl materials from NSM Neoresins, and the AC and AS polymers Alberdingk-Boley.

Various additives for controlling or regulating the pH of the inkjet ink composition of the present invention may also be used. Examples of suitable pH regulators include various amines such as diethanolamine and triethanolamine as well as various hydroxide reagents. An hydroxide reagent is any reagent that comprises an OH⁻ ion, such as a salt having an hydroxide counterion. Examples include sodium hydroxide, potassium hydroxide, lithium hydroxide, ammonium hydroxide, and tetramethyl ammonium hydroxide. Other hydroxide salts, as well as mixtures of hydroxide reagents, can also be used. Furthermore, other alkaline reagents may also be used which generate OH⁻ ions in an aqueous medium. Examples include carbonates such as sodium carbonate, bicarbonates such as sodium bicarbonate, and alkoxides such as sodium methoxide and sodium ethoxide. Buffers may also be added.

Additionally, the inkjet ink composition of the present invention may further incorporate conventional dyes to modify color balance and adjust optical density. Such dyes include food dyes, FD&C dyes, acid dyes, direct dyes, reactive dyes, derivatives of phthalocyanine sulfonic acids, including copper phthalocyanine derivatives, sodium salts, ammonium salts, potassium salts, lithium salts, and the like.

The inkjet ink composition can be purified and/or classified using methods such as those described above for the modified pigments of the present invention. An optional counterion exchange step can also be used. Thus, unwanted impurities or undesirable large particles can be removed to produce an ink with good overall properties.

The present invention will be further clarified by the following examples, which are intended to be purely exemplary of the present invention.

### EXAMPLES

### Example 1

A mixture of 10.3 grams pigment blue 15:4 (available from Sun Chemicals, oven dried from presscake), 2.1 grams maleic anhydride, and 100 milliliters tetrahydrofuran was prepared and mixed with a rotor-stator for 10 minutes. The mixture was then evaporated until dry. Solids were scraped from the flask and heated for six hours at 180 degrees C. After cooling to room temperature, the solids were dispersed in 50 milliliters 1.0N KOH via 20 minutes of sonication at 0 degrees C. in a Misonix 3000 sonicator using the microtip at a power setting of 5.5 and a pulse cycle of 8 seconds on, 2 seconds off. After sonication, no particles were observed (via visual inspection) to settle out of suspension. After 24 hours the dispersion had still not settled. The dispersion was then sonicated for another 20 minutes (at the same settings), prior to measuring a particle size. A volume-weighted mean particle size of 422 nanometers was measured using a Microtrac® Particle Size Analyzer dynamic light scattering device. The dispersion was further purified by diafiltration with 10 volumes deionized water using Spectrum Membrane (1050 cm²) and a peristaltic pump. Three days later, a volume-weighted mean particle size of 556 nanometers was measured, indicating that the particles had not grown substantially in three days and that the aqueous dispersion including the modified pigment was essentially stable. A zeta potential of −42 millivolts was measured using a Brookhaven Zeta Plus, indicating the presence of a strong negative charge on the dispersed particles. The measured zeta-potential further indicates that the dispersion was essentially stable.

### Example 2

A mixture of 46 grams pigment blue 15:4 (available from Sun Chemicals, oven dried from presscake), 9.2 grams maleic anhydride, and 400 milliliters tetrahydrofuran was prepared and mixed with a rotor-stator for 10 minutes. The mixture was then evaporated until dry. Solids were scraped from the flask and heated for six hours at 180 degrees C. and cooled to room temperature. The solids were utilized in Examples 3 and 6 as described in more detail below.

### Example 3

A mixture of 5 grams of the solids prepared in Example 2 and 60 grams ethylene diamine was prepared and sonicated 40 minutes at 0 degrees C. (according the sonicating procedure described in Example 1). A volume-weighted mean particle size of 840 nanometers was measured using a Microtrac® Particle Size Analyzer. The particle size distribution was bi-modal, having peaks at approximately 400 and 1500 nanometers.

### Example 4

A mixture of methacrylic acid/methyl methacrylate/benzyl methacrylate (25% methacrylic acid, 70% butyl acrylate, 5% benzyl methacrylate) was prepared using semi-continuous feed techniques at 50% solids in a 50/50 blend of butanol and isobutyl acetate. Dodecanethiol (2.5% based on the monomer feed) and butoxyethyl acetate (3% based on the monomer feed) were added to the mixed monomers. The mixture was fed into the solvent over a period of 170 minutes with the temperature being held between 85 and 95 degrees C. Benzoyl peroxide (1% based on the monomer feed) was dissolved in a small amount of N-methylpyrrolidone and added as a co-feed over the same period of time. The reaction temperature was maintained for one hour after completion of the benzoyl peroxide feed. Another aliquot of benzoyl peroxide, equal to the first, was added at the end of the hour. The polymer was twice collected by precipitation into hexanes and then dissolved in tetrahydrofuran (at approximately 10-20% solids). The inherent viscosity of the polymer in the tetrahydrofuran was measured to be 0.09 dL/g and the acid number was 145 milligrams KOH per gram of polymer.

### Example 5

Anhydrous N-methylpyrrolidone (180 grams) was charged to a three-neck, 500 milliliter round bottom flask equipped with a spin bar, condenser, and nitrogen inlet. Twenty (20) grams was added and dissolved in the N-methylpyrrolidone with stirring. The contents of the flask were heated to 70 degrees C. using a thermostatted oil bath. Ethylene diamine (0.24 gram) was added in one charge to the stirring polymer solution. After five minutes, 4.96 grams of a 1M solution of dicyclohexylcarbodiimide in dichloromethane was added to the reactor in a single charge. The reaction was held at 70 degrees C. for three hours, cooled to ambient temperature, and stirred an additional 14 hours. A small amount of white precipitate formed and was removed via vacuum filtration. The resulting polymer solution was used without further purification.

### Example 6

The modified polymer of Example 5 (2 grams polymer in 10 milliliter N-methylpyrrolidone), 2 grams of the solids prepared in Example 2, and 5 milliliter N-methylpyrrolidone were combined and sonicated (as described in Example 1) at 0 degrees C. for 30 minutes. The mixture was then heated in an oven to 60 degrees C. for three days. At the end of the three days, the sample was poured into 200 milliliters 100 mM NaOH and sonicated for 7 minutes. A volume-weighted mean particle size of 200 nanometers was measured using a Microtrac® Particle Size Analyzer. The dispersion was diafiltered 5 volumes 50 mM NaCl and then in 10 volumes DI water. A final volume-weighted mean particle size of 311 nanometers was measured, with a peak in the distribution at 200 nanometers.

The foregoing description of preferred embodiments of the present invention has been presented for the purposes of illustration and description. It is not intended to be exhaustive or to limit the invention to the precise form disclosed. Modifications and variations are possible in light of the above teachings, or may be acquired from practice of the invention. The embodiments were chosen and described in order to explain the principles of the invention and its practical application to enable one skilled in the art to utilize the invention in various embodiments and with various modifications as are suited to the particular use contemplated. It is intended that the scope of the invention be defined by the claims appended hereto, and their equivalents.

What is claimed is:

1. A modified colored pigment comprising:
a colored pigment; and
at least one attached organic group;
wherein the organic group is attached to the colored pigment via at least one carbon atom of a C—C single bond or double bond, wherein the C—C single bond or double bond is not a component of an aromatic system, and wherein the organic group further comprises at least one activating group on at least one carbon atom of the C—C single bond or double bond.

2. The modified colored pigment of claim 1, wherein the activating group comprises an electron withdrawing group.

3. The modified colored pigment of claim 1, wherein the activating group is a carboxyl group, an alkoxycarbonyl group, an aryloxycarbonyl group, an acyloxycarbonyl group, a carbamoyl group, an alkylcarbamoyl group, an arylcarbamoyl group, or an acylcarbamoyl group.

4. The modified colored pigment of claim 1, wherein the activating group is selected from the group consisting of —CO—R⁵, —CHO, —COOR⁵, —COOH, —CN, —SO₂R⁵, and —CO—X—CO—, wherein X is selected from the group consisting of: O and N—R⁵; and wherein R⁵ is selected from the group consisting of: alkyl, Y-functionalized alkyl, polymers, cyclic organic groups, aryl, and Y-functionalized aryl of the form Ar—Yₙ; wherein Y is selected from the group consisting of: —OH, —OR, —SH, —SR, —SO₃H, —SO₃⁻M⁺, —SO₂R, —B(OH)₂, —O(CH₂—CH₂—O)ₘ—H, —COOH, —COO⁻M⁺, —NH₂, —NR₂, —N((CH₂—CH₂—O)ₙH)₂, —CON((CH₂—CH₂—O)ₙH)₂, trialkoxysilyl, perfluoroalkyl, alkyl, —NH₃⁺Q⁻, —NR₃⁺Q⁻, —SO₂—NR₂, —NO₂, —Cl, —COR, —COOR, —NO₂, —SSR, —SCN, —CN, —PO₃H₂, and —PO₃⁻²M⁺₂; M is an alkali metal ion or an alkaline earth metal ion, Q⁻ is a halide ion, a nitrate ion, a carboxylate ion, or a sulfate ion; m is 1 to 50; and n is from 1 to 5.

5. The modified colored pigment of claim 4, wherein the polymers are polyamines, polyalkylene oxides, polyalkylene glycols, polyols, poly(meth)acrylates, proteins, polyamino acids, polyureas, polyurethanes, polyureas/urethanes, polyesters, polyethers, poly-vinylethers, polyamides, polyimides, polyolefins, polyolefin-acrylates, polystyrenes, styrene(meth)acrylate copolymers, salts thereof, or derivatives thereof.

6. The modified colored pigment of claim 1, wherein the colored pigment is black, blue, brown, cyan, green, white, violet, magenta, red, orange, or yellow pigment, or a mixture thereof.

7. The modified colored pigment of claim 1, wherein the colored pigment is an aniline black, a perylene black, an anthraquinone, a phthalocyanine blue, a phthalocyanine green, a disazo pigment, a monoazo pigment, a pyranthrone, a heterocyclic yellow, a quinacridone, a quinolonoquinolone, a (thio)indigoid, or a mixture thereof.

8. The modified colored pigment of claim 1, wherein the colored pigment is a phthalocyanine blue, a quinacridone, or a mixture thereof.

9. The modified colored pigment of claim 1, wherein the organic group is attached to the colored pigment in an amount sufficient to improve the dispersibility of the surface-modified colored pigment in an aqueous medium.

10. A modified colored pigment comprising the reaction product of a nucleophilic polymer and the modified colored pigment of claim 1.

11. The modified colored pigment of claim 10, wherein the nucleophilic polymer comprises or is modified to comprise a nucleophile, wherein the nucleophilic polymer is selected from the group consisting of polyamines, polyalkylene oxides, polyalkylene glycols, polyols, poly(meth)acrylates, proteins, polyamino acids, polyureas, polyurethanes, polyureas/urethanes, polyesters, polyethers, poly-vinylethers, polyamides, polyimides, polyolefins, polyolefin-acrylates, polystyrenes, styrene(meth)acrylate copolymers, salts thereof, and derivatives thereof.

12. A modified colored pigment comprising the reaction product of:
a colored pigment; and
at least one organic compound comprising a C—C double or triple bond and at least one activating group on at least one carbon atom of the C—C double or triple bond.
wherein the reaction product comprises an organic group attached to the colored pigment via at least one carbon atom of a C—C single bond or double bond, wherein the C—C single bond or double bond is not a component of an aromatic system, and wherein the organic group further comprises the activating group on at least one carbon atom of the C—C single bond or double bond.

13. The modified colored pigment of claim 12, wherein the organic compound is maleic anhydride, maleic acid, acetylene dicarboxylic acid, fumaric acid, or derivatives of maleic acid, acetylene dicarboxylic acid, or fumaric acid, wherein the derivatives are carboxylic acids, dicarboxylic acids, esters, amides, or imides.

14. The modified colored pigment of claim 12, wherein the organic compound is maleic anhydride.

15. The modified colored pigment of claim 12, wherein the activating group is a carboxyl group, an alkoxycarbonyl group, an aryloxycarbonyl group, an acyloxycarbonyl group, a carbamoyl group, an alkylcarbamoyl group, an arylcarbamoyl group, or an acylcarbamoyl group.

16. The modified colored pigment of claim 12, wherein the colored pigment is an aniline black, a perylene black, an anthraquinone, a phthalocyanine blue, a phthalocyanine green, a disazo pigment, a monoazo pigment, a pyranthrone, a heterocyclic yellow, a quinacridone, a quinolonoquinolone, a (thio)indigoid, or a mixture thereof.

17. The modified colored pigment of claim 12, wherein the colored pigment is a phthalocyanine blue, a quinacridone, or a mixture thereof.

18. A dispersion comprising:
a liquid vehicle; and
a modified colored pigment dispersed in the liquid vehicle,
wherein the modified colored pigment comprises a colored pigment and at least one attached organic group, the organic group being attached to the colored pigment via at least one carbon atom of a C—C single bond or double bond, wherein the C—C single bond or double bond is not a component of an aromatic system, and wherein the organic group further comprises at least one activating group on at least one carbon atom of the C—C single bond or double bond.

19. The dispersion of claim 18, wherein the liquid vehicle is an aqueous liquid vehicle.

20. The dispersion of claim 18, wherein the liquid vehicle is a non-aqueous liquid vehicle.

21. An ink jet ink comprising:
a liquid vehicle; and
a modified colored pigment dispersed in the liquid vehicle,
wherein the modified colored pigment comprises a colored pigment and at least one attached organic group, the organic group being attached to the colored pigment via at least one carbon atom of a C—C single bond or double bond, wherein the C—C single bond or double bond is not a component of an aromatic system, and wherein the organic group further comprises at least one activating group on at least one carbon atom of the C—C single bond or double bond.

22. The ink jet ink of claim 21, wherein the liquid vehicle is an aqueous liquid vehicle.

23. The ink jet ink of claim 21, wherein the liquid vehicle is a non-aqueous liquid vehicle.
